(19) United States
(12) Patent Application Publication
(10) Pub. No.: US 2026/0048281 A1
(43) Pub. Date: Feb. 19, 2026

(54) SYSTEMS AND METHODS FOR MODULATING DEFAULT MODE NETWORK BRAIN ACTIVITY

(71) Applicant: ATTUNE NEUROSCIENCES, INC., Menlo Park, CA (US)

(72) Inventors: Keith R. Murphy, San Francisco, CA (US); Joline M. Fan, San Francisco, CA (US); Cara Woodworth, San Francisco, CA (US); Andrew D. Krystal, San Francisco, CA (US)

(73) Assignee: ATTUNE NEUROSCIENCES, INC., Menlo Park, CA (US)

(21) Appl. No.: 19/231,038

(22) Filed: Jun. 6, 2025

Related U.S. Application Data
(60) Provisional application No. 63/657,683, filed on Jun. 7, 2024.

(51) Int. Cl. A61N 7/00

(52) U.S. Cl. CPC A61N 7/00; A61N 2007/0026; A61N 2007/0078

(57) ABSTRACT

The present disclosure relates to systems, devices, and methods for ultrasound neuromodulation of a target brain region to modulate the connectivity within the default mode network. The method involves administering focused ultrasound configured to modulate the connectivity within the default mode network. Modulating the connectivity of the default mode network can promote mental well-being, decrease the frequency and duration of non-present-focused thinking, enhance present-tense experiential engagement, improve attention, reduce mind wandering during tasks, and/or mitigate ruminative thinking. The focused ultrasound can target the anterior thalamus or neighboring structures, utilizing two transducers placed over the temporal window to create a cross beam overlapping the thalamus. Ultrasound may be pulsed within a spectral range of 10-30 Hz, with a duty cycle of less than 15%, and delivered over a period of at least 5 minutes.

### CROSS-REFERENCE TO RELATED APPLICATION

This application claims the benefit of U.S. Provisional Application No. 63/657,683, filed Jun. 7, 2024, which is incorporated by reference herein in its entirety.

### FIELD

The present disclosure generally relates to devices, and associated systems, methods, and uses for modulating brain activity using ultrasound stimulation.

### BACKGROUND

Focused ultrasound (FUS) is recognized as a potent tool for non-invasive modulation of intact brain circuits. It is particularly advantageous since, unlike pharmacologic or other existing forms of non-invasive brain stimulation approaches, it can be used to target specific, deep regions of the brain with high spatial and temporal precision. Many existing brain stimulation approaches are invasive, requiring implantation of stimulative electrodes into target regions of the brain to provide therapeutic effects, while non-invasive techniques are unable to target deeper subcortical regions.

The Default Mode Network (DMN) is a critical highly connected network between regions of the brain which supports the complex interplay between self-awareness and consciousness. The DMN is composed of the medial prefrontal cortex, posterior cingulate cortex, and the angular gyri. This network is most active when an individual is not focused on the external environment, engaged in activities such as daydreaming, recalling memories, or contemplating the future. The thalamus, a central neural relay station, is intricately linked with the DMN, playing a pivotal role in coordinating and integrating brain activities. Its extensive connections enable the thalamus to potentially regulate the DMN's synchronization, crucial for maintaining resting state functions and transitioning to task-focused states.

Many conventional approaches to modulating the activity of the default mode network require invasive techniques, while many conventional non-invasive techniques are unable to target deeper subcortical regions.

The embodiments disclosed herein are directed to addressing these and other considerations.

### SUMMARY

According to aspects of the present disclosure, a head-mounted device designed to modulate brain activity through focused ultrasound is disclosed. The device may include an ultrasound transducer element housing that accommodates one or more ultrasound transducer elements. These elements may be positioned near the temporal window of a user when the device is worn on the head. The device may include one or more processors and a non-transitory memory in operative communication with the one or more processors. When executed, the instructions are configured to cause the one or more processors to control the ultrasound transducer elements to create at least one ultrasound focus at a target region within the user's head. The focused ultrasound delivered to the target region can operate at a frequency between approximately 100 kHz and 10 MHz, thereby modulating the level of connectivity within the user's default mode network.

The target brain region may include at least a portion of the thalamus. In some embodiments, the target region includes the anterior thalamus. Modulating the connectivity within the default mode network can increase present-tense experiential engagement by reducing non-present focused thinking, enhance attention during tasks, and decrease ruminative thinking. The ultrasound transducer elements may include a first and a second element, each positioned within the housing at different locations. The non-transitory memory may contain further instructions for the one or more processors to control these elements, causing them to deliver focused ultrasound beams that intersect at a predetermined angle at the target region.

In some embodiments, the device may be configured to pulse the ultrasound transducer elements at a pulse rate between approximately 10 Hz and 30 Hz. In some embodiments, the device may be configured to deliver focused ultrasound with a duty cycle of less than 15%. In some embodiments, the device may be configured to deliver a first pulsing pattern for a duration of at least five minutes.

According to some embodiments, modulating the default mode network connectivity may also lead to a reduction in symptoms associated with major depressive disorder, obsessive-compulsive disorder, anxiety, post-traumatic stress disorder, attention deficit hyperactivity disorder, autism spectrum disorder, sleep disorders, or any combination thereof.

In another aspect, the present disclosure describes a method of providing focused ultrasound to a target region of the user's head. The method may include creating an ultrasound focus at a target region within the user's head and delivering focused ultrasound to modulate connectivity within the default mode network. The method may include positioning of the transducer elements near the temporal window, targeting the anterior thalamus, and achieving benefits to experiential engagement, attention, and reduction of ruminative thinking.

In some embodiments, the method may include pulsing the ultrasound transducer elements at a pulse rate between approximately 10 Hz and 30 Hz. In some embodiments, the method may include delivering focused ultrasound with a duty cycle of less than 15%. In some embodiments, the method may include delivering a first pulsing pattern for a duration of at least five minutes.

### BRIEF DESCRIPTION OF THE DRAWINGS

The present disclosure can be better understood, by way of example only, with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1A illustrates a neuromodulation device and a stimulation control computing environment according to aspects of the present disclosure.

FIG. 1B illustrates exemplary hardware components of a neuromodulation device according to aspects of the present disclosure.

FIG. 1C illustrates an exemplary application of focused ultrasound to a target brain region, according to aspects of the present disclosure.

FIG. 2 depicts effects of applying focused ultrasound to various target brain regions, according to aspects of the present disclosure.

FIG. 3A depicts an exemplary experimental design for measuring the effects of focused ultrasound, according to aspects of the present disclosure.

FIG. 3B depicts an overlay of focused ultrasound over images of various target brain regions and a control, according to aspects of the present disclosure.

FIG. 3C depicts effects on subject depression scores after exposing various target brain regions to focused ultrasound, according to aspects of the present disclosure.

FIG. 3D depicts effects on subject depression scores as a function of time, according to aspects of the present disclosure.

FIG. 3E depicts effects on subject depression scores after exposing various target brain regions to focused ultrasound, according to aspects of the present disclosure.

FIG. 3F depicts effects on subject depression scores as a function of time, according to aspects of the present disclosure.

FIG. 3G depicts default mode network connectivity before and after application of focused ultrasound, according to aspects of the present disclosure.

FIG. 3H depicts default mode network connectivity before and after application of focused ultrasound, according to aspects of the present disclosure.

FIG. 4A illustrates default mode network connectivity before and after application of focused ultrasound, according to aspects of the present disclosure.

FIG. 4B depicts a relationship between default mode network connectivity and applied pressure of focused ultrasound, according to aspects of the present disclosure.

FIG. 5A depicts a relationship between energy levels of subjects before and after application of focused ultrasound, according to aspects of the present disclosure.

FIG. 5B depicts a relationship between focus levels of subjects before and after application of focused ultrasound, according to aspects of the present disclosure.

FIG. 6 depicts a relationship between subject mood in healthy individuals and an application of focused ultrasound, according to aspects of the present disclosure.

### DETAILED DESCRIPTION

The present disclosure is related to systems, devices, and methods for providing focused ultrasound to a target brain region of a user with a head-worn neuromodulation device. Embodiments consistent with the present disclosure address the need for devices, systems, and methods for modulating the connectivity of the default mode network (DMN) of a user based on the application of focused ultrasound to a target brain region. The target brain region can include a portion of the thalamus, and more particularly, the anterior thalamus (ANT). Based on an application of focused ultrasound to the target brain region, the disclosed devices, systems, and methods are configured to modulate the connectivity of the default mode network. According to some aspects of the present disclosure, modulating the connectivity of the default mode network increases present-tense experiential engagement by decreasing frequency or duration of non-present focused thinking. According to some aspects of the present disclosure, modulating the connectivity of the default mode network enhances attention during tasks. According to some aspects of the present disclosure, modulating the connectivity of the default mode network results in a reduction of ruminative thinking. According to some aspects of the present disclosure, modulating the connectivity of the default mode network can result in a reduction in symptoms of major depressive disorder, obsessive-compulsive disorder, anxiety, post-traumatic stress disorder, attention deficit hyperactivity disorder, autism spectrum disorder, sleep disorder, or any combination thereof.

The neuromodulation system can comprise a wearable neuromodulation device integrated with EEG electrodes and one or more integrated ultrasound transducer arrays. The disclosed neuromodulation system can further include a stimulation control unit comprising one or more processors, and software that operates and controls the features and functionality of the ultrasound stimulation when executed by such processors. Such software includes, without limitation, EEG real-time analysis software that can continuously monitor brain functionality to identify one or more certain characteristics, phases or states of brain activity, and brain mapping software that can plot one or more specific region of the brain and accurately focus or steer ultrasound stimulation to that one or more specific brain regions. The disclosed neuromodulation system also includes a computational device that aids in neuromodulation device operation and data storage of collected information. Thus, a neuromodulation system disclosed herein noninvasively administers ultrasound stimulation in a spatially and temporally controlled manner. As such, a device disclosed herein enables a focus application of ultrasound stimulation to a specified region of the brain that largely excludes surrounding brain tissue.

The disclosed neuromodulation device can be coupled to brain substructure mapping software that identifies one or more specific regions to be targeted for ultrasound stimulation. In some embodiments, one or more specific regions of the brain are identified by comparing a brain image scan to a brain atlas which may be publicly available or internally annotated to identify a common coordinate space. The system can use brain image scans, including scans generated by computed tomography (CT) and magnetic resonance imaging (MRI) to determine a position of the one or more transducer elements relative to the temple of a user. Non-limiting sources of such brain image scans include scans obtained from a user of a neuromodulation device disclosed herein (personalized model customized for a particular user), scans obtained from deidentified individuals through healthcare facilities, or scans obtained from deidentified individuals through registries like the Human Connectome. Brain image scans are registered with a common brain region atlas and image segmentation performed to identify centroids in voxel space of the one or more specific regions to be targeted for ultrasound stimulation. In some embodiments, the target brain region is the thalamus. In some embodiments, the one or more specific regions to be targeted for ultrasound stimulation is a sub-region of the thalamus such as, but not limited to, the anterior thalamus and the mediodorsal thalamus (DMT). In some embodiments, the one or more specific regions to be targeted with ultrasound stimulation is the ventral capsule (VC), the anterior thalamus, the mediodorsal thalamus, the bed nucleus of the stria terminalis (BNST), or any combination thereof.

In some embodiments, the position of one or more ultrasound transducer elements relative to the temporal window (e.g., temple of a user) can be identified using biometric parameters (e.g., fiducial landmark elements). The position of the one or more transducers relative to the temporal window (e.g., the temple of a user) may be estimated as a point in 3D space relative to a fiducial landmark element which have some predictive value for the positioning of the ultrasound transducer elements relative to the temporal window. This biometric may include, but is not limited to, the position of a person's eyes, ears, eyebrow ridge, nose, mouth, jawline, or other appendage relative to a cranial landmark. This biometric may also include a relative point along a cranial feature axis, such as a fractionally defined mid-point along the forehead, between the ears and eyes, between the corner of the mouth to the base of the ear, or some other combination of cranial features or appendages.

Once the one or more specific target brain regions are identified, a brain substructure mapping software disclosed herein can identify the coordinate space of each transducer elements within the image data. According to some embodiments, the system can register the initial position of the one or more ultrasound transducer elements relative to the temporal window using one or more fiducial landmark elements as described above. The system may then accurately calculate the temporal phase offset of ultrasound transducer elements by estimating acoustic temporal path length between the element and the target brain regions of one or more identified locations or by performing a full wave simulation. Initially the software may determine acoustic impedance by employing an algorithm that converts pixels of a brain image scan from the brain modeling database into measurements of acoustic impedance (Hounsfield units). A brain substructure mapping software may determine the appropriate phase of ultrasound emissions form the one or more ultrasound transducer elements required to effectively apply ultrasound stimulation onto a target brain region. In some embodiments, the required beam steering is determined by modeling simulations of wave equations by estimating the temporal wave path length to the target brain area, accounting for difference in sound speed across skull and tissue as well as wave refraction. The simulation then adjusts the excitation phase delay of each ultrasound transducer element until the wave fronts constructively interfere at the focus. This process may be referred to herein in shorthand as computing and applying a phase change to ultrasound emissions to create an ultrasound focus at a target brain region.

Once the initial position of the one or more ultrasound transducer elements relative to the temporal window is determined, the neuromodulation device can accurately focus ultrasound emissions from the one or more ultrasound transducer elements to the target brain region. By applying FUS to one or more target brain regions, the disclosed devices, systems, and methods are configured to modulate the connectivity of the default mode network, as described herein.

Accordingly, embodiments of the present disclosure solve the long-standing problem of providing a non-invasive therapeutic effect to users with conditions such as, but not limited to major depressive disorder, obsessive-compulsive disorder, anxiety, post-traumatic stress disorder, attention deficit hyperactivity disorder, autism spectrum disorder, sleep disorder, or any combination thereof.

In the realm of consciousness and meditation, the DMN plays a pivotal role. Consciousness, a multifaceted phenomenon, involves self-awareness and the perception of external and internal stimuli. The DMN's activity is closely linked to self-referential thought processes—a key component of consciousness. Meditation, a practice aimed at achieving a heightened state of awareness and focused attention, has been shown to modulate the activity of the DMN. Various forms of meditation, such as mindfulness and focused-attention meditation, have been associated with decreased activity in the DMN, correlating with a reduction in mind-wandering and enhanced present-moment awareness. These findings suggest that the DMN is not only a marker of the wandering mind but also a potential target for practices aimed at enhancing consciousness and cognitive control.

Disruptions in the DMN have been implicated in a range of disorders, underlining its significance in mental health. Alterations in the functionality and connectivity of the DMN have been observed in conditions such as depression, post-traumatic stress disorder (PTSD), schizophrenia, attention deficit hyperactivity disorder (ADHD), autism, obsessive compulsive disorder (OCD), and Alzheimer's disease. These disruptions often manifest as aberrant activity levels in the network, leading to symptoms like excessive rumination, impaired attention, and deficits in memory and executive function. The study of these alterations provides valuable insights into the pathological mechanisms underlying these disorders and highlights the DMN's role in maintaining cognitive and emotional well-being.

The modulation of the DMN has become a focal point in developing treatments for various psychological and neurological disorders. Pharmacological interventions, psychotherapy, and mind-body practices such as meditation have been explored for their effects on the DMN. For instance, antidepressants have been shown to normalize DMN activity in depressed patients, correlating with symptom improvement. Similarly, cognitive behavioral therapy has demonstrated efficacy in modulating DMN activity, particularly in anxiety disorders. Additionally, mindfulness-based interventions, by altering DMN activity, have shown promise in reducing symptoms of depression and anxiety, as well as improving cognitive function in aging populations. These treatment modalities highlight the therapeutic potential of targeting the DMN, offering new avenues for intervention and a deeper understanding of the brain's intricate workings. However, mindfulness-based interventions are difficult for users to maintain, and pharmacological interventions often cause unwanted side effects to users of pharmacological interventions. Further, although invasive techniques such as deep brain stimulation (DBS) have shown promise in modulating the activity of the default mode network, many users are unwilling to undergo such an invasive procedure. Furthermore, stability of optimal long-term treatment using DBS may be challenged by dynamic neuroplastic adaptations. Further, conventional noninvasive approaches, such as transcranial magnetic stimulation (TMS) are unable to target deeper subcortical regions.

The interaction between the thalamus and the default mode network is key in understanding the dynamics of consciousness, as both structures are involved in cognitive processes and conscious awareness. Furthermore, disruptions in thalamic and DMN interactions are observed in various neurological and psychiatric disorders, suggesting a shared pathophysiological mechanism. The modulation of thalamic activity, through interventions like deep brain stimulation or mindfulness practices, offers promising avenues for influencing DMN activity, which is beneficial in treating disorders associated with DMN dysregulation. This complex relationship underscores the significance of the thalamus-DMN interplay in the broader understanding of brain function and mental health.

The anterior thalamus (ANT) serves pivotal roles in cognition, encompassing a few primary functions. First, it contributes significantly to memory and learning processes, particularly in consolidating and retrieving memories associated with spatial navigation and episodic contexts. Notably, lesions in the ANT have been found to impair spatial working memory, demonstrated through tasks like the 8-arm radial maze. Additionally, the ANT contributes to arousal/attention and executive processing, modulating alertness, attentional resource allocation, and decision-making processes. The role of the ANT in attention is likely modulated, at least in part, via reciprocal connections between the ANT and the prefrontal cortex (PFC). Intriguingly, dysregulation of the ANT has also been linked to depression.

Adjacent to the ANT, the mediodorsal thalamus (DMT) also plays a pivotal role in cognition. The DMT is known to contribute to executive processing by virtue of its extensive connections with the prefrontal cortex (PFC), influencing goal-directed behavior and cognitive control. Moreover, the DMT is heavily involved in working memory, facilitating the maintenance and updating of information crucial for recall and comprehension, with connections to the PFC. Notably, lesions in the DMT have been observed to impair go/no-go reward value discriminations and recency memory but have not been shown to have significant impact on spatial memory processing. Additionally, the DMT contributes to attentional control through projections from the reticular formation and to the PFC and other cortical areas, with dysfunction leading to deficits in attention and vigilance. Furthermore, the DMT's extensive connections to the PFC underlie its role in decision-making, as aberrant functioning of the DMT can impair the capacity to evaluate options and make choices. While its role in emotional regulation is subordinate to cognitive processing, the DMT integrates emotional information with cognitive processing via connections to the amygdala and hypothalamus, thereby influencing emotional responses downstream.

Accordingly, modulation of the default mode network is implicated across a variety of psychiatric disorders, such as ADHD, OCD, autism spectrum disorder (ASD), schizophrenia, and insomnia. Aspects of the present disclosure are directed to modulation of the default mode network based on an application of FUS to one or more target brain regions.

### Modulation of the DMN and ADHD

ADHD is a neurodevelopmental disorder marked by attentional impairments, hyperactivity, and impulsivity. It is one of the most common neurodevelopmental disorders, affecting an estimated 10% of children and adolescents in the United States. Children with ADHD have worse educational outcomes and carry an associated 5× greater economic burden in comparison to children without ADHD. Symptoms often improve in adulthood; however, adults living with ADHD are 60% more likely to be fired, earn 33% less, and represent an estimated annual economic burden of $123 billion in the United States.

The main circuitry implicated in ADHD pathophysiology is altered fronto-striato-pallido-thalamo-cortical connectivity. This circuit encompasses connections between the frontal cortex, striatum, globus pallidus, thalamus, and back to the cortex. Dysfunctionality within this circuit contributes to deficits in impulse control, attention regulation, and working memory. Traditionally, drugs targeting norepinephrine (NE) and dopamine (DA) systems have been employed to treat ADHD symptomatology. First pass treatment typically involves psychomotor stimulants including methylphenidate and amphetimine, followed by nonstimulants including the NE reuptake inhibitor atomexetine and the NE agonists guanfacine and clonidine. However, stimulant medications are ineffectual in ˜30% of ADHD patients, and non-stimulant pharmacological interventions often have side effects including loss of appetite, nausea, irritability, and insomnia. Several of the neural circuits implicated in ADHD pathophysiology are also incorporated into the default mode network (DMN). Indeed, multiple imaging studies have found circuit alterations in the DMN in people diagnosed with ADHD. An inability to suppress the activity and intrusion of the DMN during attentionally-demanding tasks is believed to be a core contributor to attentional dysfunction observed in ADHD, which is known as the DMN interference hypothesis of ADHD. Thus, non-invasive manipulation and specifically inactivation of the DMN may be a useful and effective intervention towards the treatment and amelioration of ADHD symptoms.

### Modulation of the DMN and OCD

The Cortico-Striato-Thalamo-Cortical (CSTC) circuit is widely recognized as central to the pathophysiology of OCD. This complex network includes interconnected regions such as the orbitofrontal cortex (OFC), anterior cingulate cortex (ACC), striatum (specifically the caudate nucleus), and thalamus. Dysregulation within this circuitry is thought to be a fundamental mechanism underlying the repetitive thoughts and behaviors that characterize OCD. Abnormalities in the CSTC circuit are hypothesized to disrupt the normal flow of information, leading to the persistence of obsessive thoughts and compulsive actions.

The OFC plays a critical role in decision-making, reward processing, and response inhibition. In OCD, dysfunction in the OFC is associated with difficulties in evaluating the significance of stimuli and regulating behavioral responses. This impairment can lead to the misattribution of importance to certain thoughts or actions, thereby contributing to compulsive behaviors. The OFC's inability to properly inhibit inappropriate responses may result in the persistence of rituals and the excessive repetition of behaviors.

The ACC is involved in error detection, cognitive control, and emotional processing. In individuals with OCD, ACC dysfunction may lead to significant challenges in monitoring and regulating behavior, which contributes to the repetitive nature of OCD symptoms. The ACC's role in error detection suggests that its impairment could result in an exaggerated response to perceived mistakes, fueling the cycle of obsessions and compulsions. The basal ganglia, particularly the caudate nucleus, are implicated in the pathophysiology of OCD. This region is crucial for motor control, habit formation, and procedural learning. Dysregulation within the basal ganglia is believed to contribute to the development of compulsive rituals and repetitive behaviors seen in OCD. Abnormal activity in the caudate nucleus may disrupt the smooth execution of motor plans and contribute to the habitual nature of compulsions.

The thalamus acts as a relay station for sensory and motor information and plays a significant role in regulating attention and filtering sensory input. Abnormalities in thalamic function are thought to contribute to the intrusive thoughts and heightened sensory experiences characteristic of OCD. The thalamic inability to properly filter information may result in an overload of sensory input, which can exacerbate obsessive thinking and compulsive behavior. The serotonin neurotransmitter system has been extensively studied in the context of OCD, with dysregulation in this system implicated in the disorder. Serotonin modulates mood, anxiety, and impulse control. Alterations in serotonin signaling pathways may contribute to the obsessions and compulsions observed in OCD. Pharmacological treatments that target serotonin reuptake are among the most effective interventions for reducing OCD symptoms, underscoring the importance of this neurotransmitter system.

### Modulation of the DMN and ASD

Autism Spectrum Disorders (ASDs) encompass a complex class of neurodevelopmental disorders and afflict an estimated 1 in 36 children. ASDs are typically behaviorally defined by impairments in social behavior and communication and are often comorbid with auditory sensitivities and repetitive stereotypies. ASDs are significantly sex-biased, with 4.5 biological males receiving an ASD diagnosis for every 1 biological female. Additionally, ASDs differently between sexes, even upon the same genetic insult. Genetically, hundreds of ASD-associated genes and chromosome regions have been identified, owing to the complex and multifaceted nature of these disorders.

Mechanistically, altered excitatory-inhibitory balance of the neocortex appears to be a common underlying neural mechanism of ASDs. In terms of functional circuitry, the DMN has been shown in multiple studies to have altered connectivity in ASDs, and key regions of the DMN have been linked to deficient social cognition in patients diagnosed with ASDs. Self-referential thought, focus, and understanding are key functions of the DMN and are often affected by ASDs. In some cases, the strength of neural connections between specific DMN nodes have been shown to correlate with the degree of ASD symptomatology.

### Modulation of the DMN and Schizophrenia

In Schizophrenia, the thalamus serves as a relay station for sensory information and plays a role in regulating arousal, attention, and information processing. Abnormalities in thalamic structure and function have been implicated in schizophrenia and may contribute to sensory gating deficits and perceptual disturbances. Dysregulation of the DMN, a network of brain regions involved in self-referential thinking and introspection, has been observed in schizophrenia. Altered connectivity within the DMN may contribute to disruptions in self-monitoring, social cognition, and the sense of agency in individuals with schizophrenia.

### Modulation of the DMN and Insomnia

The default mode network (DMN) exhibits heightened activity during wakeful rest periods, including daydreaming and mind wandering, and is active during introspective thoughts about oneself, others, past experiences, and future events. It is also engaged in internal, non-task-oriented processes but deactivates during perception, language processing, and attention tasks. The DMN demonstrates negative correlations with attention networks, suggesting reciprocal modulation. Notably, the thalamus, known for its intrinsic oscillatory rhythm crucial for cognition, exhibits different oscillations during default states despite serving as a sensory input relay center. While evidence for the DMN in infancy is limited, it stabilizes by age 9-12 and shows consistency across individuals. Deactivation of the DMN during attentionally demanding tasks improves long-term memory consolidation and is highly engaged during narrative comprehension and conceptual understanding. Reductions in DMN activity may contribute to Alzheimer's disease pathology, as metabolic reductions in the DMN precede symptomatic signs. Moreover, alterations in the DMN have been linked to major depressive disorder (MDD) and attention-deficit/hyperactivity disorder (ADHD), with reduced anticorrelation between the DMN and other brain circuits observed in patients. Interestingly, sleep deprivation decreases DMN connectivity in healthy individuals, with acute sleep deprivation showing antidepressant effects in MDD, although subsequent sleep often leads to a rapid return of depressive symptoms. Functional connectivity within the DMN is strongest during resting wakefulness, a vigilance state indicative of calmness and relaxation. Conversely, DMN circuit temporal correlation is decreased during sleep stages, indicating dynamic changes in connectivity across sleep states. Interestingly, the anterior thalamus (ANT) and mediodorsal thalamus (DMT) are selectively affected in fatal familial insomnia, as supported by post-mortem and whole-brain PET/MRI studies. These insights into the DMN's role in cognition, mood disorders, and sleep patterns underscore its significance in understanding brain function and dysfunction.

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods.

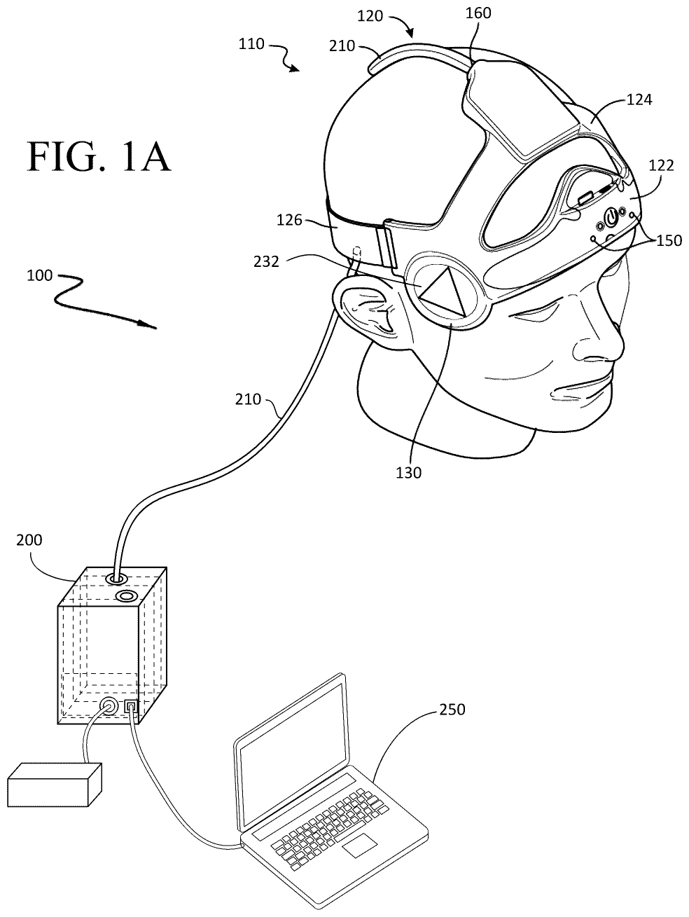

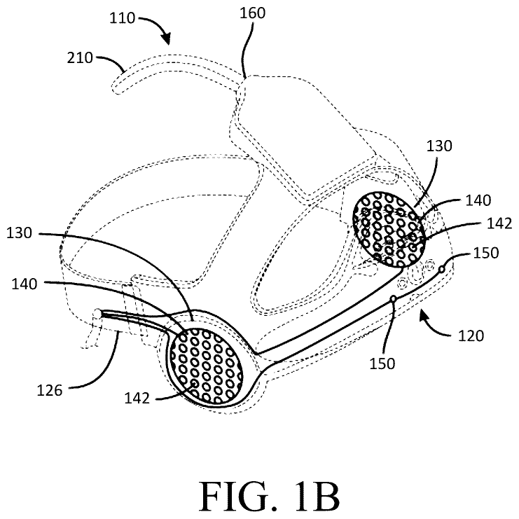

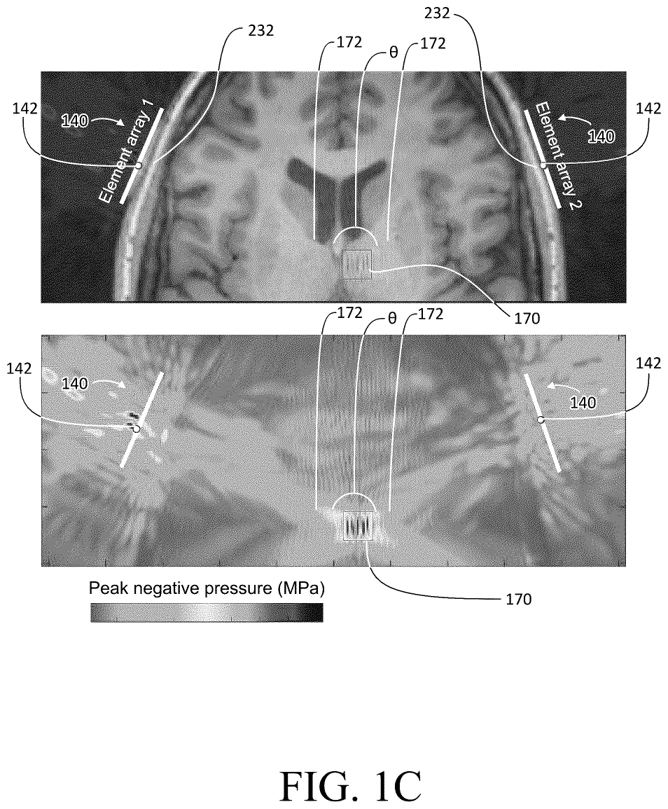

In some embodiments, and as shown in FIGS. 1A-B, an exemplary neuromodulation device 110 can include a wearable device housing 120, which can support two array housings 130 each containing an ultrasound transducer array 140, and may include two EEG electrodes 150, such as, e.g., active dry EEG electrodes. When worn, wearable device housing 120 is configured to encircle the head in a transverse plane that positions the main band along the forehead, temples and back of the head. Wearable device housing 120 provides rigid stereotactic placement of ultrasound transducer arrays 140 over the temporal window 232 of the user's head as well as positions EEG electrodes 150 flat against the user's forehead. According to some embodiments, the EEG electrodes 150 can be used to measure cortical brain activity during sleep.

Wearable device housing 120 can include a main band 122, a secondary band 124, and an optional securing strap 126. Main band 122, secondary band 124 and securing strap 126 can be adjustable to facilitate accurate positioning and securing of neuromodulation device 110 to a user's head. Secondary band 124 can be attached to main band 122 via first and secondary band attachment points and configured to extend over the top of the head. First and second band attachment points can be static or configured to allow movement between secondary band 124 and main band 122. Optional securing strap 126 is attached to main band 122 via first and second securing strap attachment points and configured to extend under the chin. First and second securing strap attachment points can be static or configured to allow movement between securing strap 126 and main band 122. In aspects of these embodiments, main band 122 has front and back portions composed of a semi-rigid material and side or temple portions composed of a flexible material, secondary band 124 and a first and second attachment hubs each being composed of a semi-rigid material, and a securing strap being composed of an elastic material. Although the embodiment shown in FIGS. 1A-B includes optional securing strap 126, it should be understood that in some embodiments, the optional securing strap 126 is omitted, and in yet other embodiments, optional securing strap 126 is replaced with a band similar to the construction of main band 122 and/or secondary band 124. Similarly, in some embodiments, main band 122 and secondary band 124 may be of unitary construction. In yet other embodiments, wearable device housing 120 can include a single band that stretches from approximate the forehead.

Neuromodulation device 110 can include one or more ultrasound transducer arrays 140 contained in a housing attached to main band 122 of wearable device housing 120. The one or more ultrasound transducer arrays 140 can be located on the inner surface of main band 122 and configured to interface with a user's head. In some embodiments, a neuromodulation device disclosed herein contains a single ultrasound transducer array 140 located on the main band. In some embodiments, neuromodulation device 110 contains a single ultrasound transducer array 140 located on one side of main band 122 positioned at either the left or right temple region of a user above the ears. In some embodiments, neuromodulation device 110 contains a single ultrasound transducer array located on each side of main band 122 positioned at the left and right temple region of a user above the ears. In some embodiments, neuromodulation device 110 contains multiple ultrasound transducer arrays 140 located on each side of main band 122 positioned at the left and right temple region of a user above the ears. In aspects of these embodiments, and as shown in FIG. 1A-B, neuromodulation device 110 comprises two ultrasound transducer arrays 140 one located on the left side of main band 122 and one located on the right side of main band 122. In aspects of these embodiments, neuromodulation device 110 comprises two ultrasound transducer arrays 140 located on the left side of main band 122 and two ultrasound transducer arrays 140 located on the right side of main band 122. It should be understood, that while each ultrasound transducer array 140 is shown having a plurality of ultrasound-emitting elements 142, in some embodiments, each ultrasound transducer array 140 can comprise a single ultrasound-emitting element 142. In some embodiments, the neuromodulation device 110 can optionally include photoplethysmography (PPG) sensors (not shown). In some embodiments, the neuromodulation device 110 can include one or more accelerometers (not shown) that may be used to capture head movement of a user wearing the neuromodulation device 110.

According to some embodiments, the ultrasound-emitting elements 142 can be designed to interface with the “temporal window” 232, a thin portion of skull bone posterior to the eyes that allows access to centralized deep brain structures. In some embodiments, the neuromodulation system 100 determines ultrasound beam steering parameters that are unique to each person's brain and skull morphology that are used to accurately target certain brain regions with FUS. In some embodiments, the neuromodulation system 100 utilizes a combination of custom automated MRI scan segmentation, transducer spatial mapping, and acoustic simulation tools to optimize off-line targeting of brain regions.

According to some embodiments, and as illustrated in FIG. 1C, a first ultrasound-emitting element 142 within an ultrasound transducer array 140 can be positioned in a first position relative to the temporal window 232 of the user, and a second ultrasound-emitting element 142 within an ultrasound transducer array 140 can be positioned in a second position relative to the temporal window 232 of the user, such that a focused ultrasound beam emitted 172 from the first ultrasound-emitting element 142 crosses with a focused ultrasound beam 172 emitted from the second ultrasound-emitting element 142 at a predetermined crossing angle θ at the target region 170 of the user. According to some embodiments, the predetermined crossing angle θ can comprise an angle between approximately 15 degrees and approximately 120 degrees. In some embodiments, the predetermined crossing angle θ can comprise an angle of approximately 90 degrees. According to certain exemplary embodiments, forming a cross-beam at the target brain region 170 increases effectiveness of the focused ultrasound at the target region 170, while reducing off-target ultrasound modulation of non-target areas (e.g., tissue areas surrounding the target region 170 of the user).

Returning to FIGS. 1A-B, neuromodulation device 110 can contain EEG electrodes 150 located on the inner surface of main band 122 and configured to interface with a user's head. In some embodiments, neuromodulation device 110 contains a single EEG electrode 150 located on the front portion of main band 122 positioned at the forehead of a user above the eyebrows. In some embodiments, neuromodulation device 110 contains multiple EEG electrodes 150, each located on the front portion of main band 122 positioned at the forehead of a user above the eyebrows. In aspects of these embodiments, and as shown in FIGS. 1A-B, neuromodulation device disclosed herein comprises two EEG electrodes 150 each located on the front portion of main band 122 with one positioned above the left eyebrow of a user and the other positioned above the right eyebrow of the user. However, it should be understood that in other embodiments, the number and specific positioning of EEG electrodes 150 can be varied.

A single EEG electrode, or a plurality of EEG electrodes comprising a neuromodulation device disclosed herein provides sufficient sensitivity to provide optimal measurement of brainwave activity, including, without limitation, wave frequency, wave amplitude, and waveform type to effectively identify one or more characteristics, phases or states of brain activity. In aspects of this embodiment, a neuromodulation device disclosed herein comprises a plurality of EEG electrodes having sufficient sensitivity to detect and measure alpha waves, beta waves, theta waves, delta waves, gamma waves, sleep spindles, K complexes, or any combination thereof.

Neuromodulation device 110 can comprise a planar, open-curved arc, or closed-curved arc configuration of EEG electrodes. The planar, open-curved arc, or closed-curved arc configuration of EEG electrode is a configuration designed to provide optimal measurement of brainwave activity, including, without limitation, wave frequency, wave amplitude, and waveform type to effectively identify one or more characteristics, phases or states of brain activity. In some embodiments, a neuromodulation device disclosed herein is a one-dimensional planar, curved or closed curved arc configuration of EEG electrodes. In some embodiments, each EEG electrode can be controlled in isolation, or in clusters to reduce cabling.

A neuromodulation device disclosed herein further contains conductive wiring. Such conductive wiring can be located exteriorly on the device housing or embedded within wearable device housing 120, such as, e.g., within a channel, and will exit the housing through a port located at the back. In some embodiments, the conductive wiring will exit cable port 160 parallel to the head in the anterior-posterior direction allowing the user to lay on his back against the flush wires. Conductive wiring disclosed herein powers an EEG amplification stage for each EEG electrode 150, each ultrasound transducer array 140, stimulation control unit 200 and its associated processing elements and functions, and other components of neuromodulation device 110 and can be bundled together. In some embodiments, conductive wiring runs through a channel within main band 122 connecting each EEG electrode 150 to one or more amplifiers, a digital analog converter, and a stimulation control unit 200 before exiting via cable port 160 located at a back portion of main band 122. In some embodiment, and with respect to each ultrasound transducer array 140, conductive wiring runs through a channel within main band 122 connecting each ultrasound transducer array 140 to stimulation control unit 200 before exiting via cable port 160 located at a back portion of main band 122.

Aspects of the present specification disclose a neuromodulation system comprising a stimulation control computing environment including a stimulation control unit and a computing device. Referring to FIG. 1A, neuromodulation system 100 further contains a stimulation control unit 200 located on main band 122 or tethered to main band 122 with conductive wiring 210 via a cable port 160. Stimulation control unit 200 comprising a central control application-specific integrated circuits (ASIC) processor, a printed circuit board (PCB) component which contains an ultrasound phase control component, one or more signal amplifiers, an ultrasound matching network as well as a power source and other processors. The ASIC chip processes EEG data, ultrasound state data, ultrasound-emitting element target phase data, power usage, and data storage. This ASIC processor sends information regarding element phase which triggers the ultrasound phase control component and one or more signal amplifiers of the PCB component. This PCB component then sends signals to the ultrasound matching network to reduce reflections from acoustic impedance mismatch and then to each ultrasound-emitting element 142 of ultrasound transducer array 140, which allow for beam steering on neuromodulation device 110. Stimulation control unit 200 uses an input file regarding phase delays for each target structure, which can be subdivisions of a single target as well as a stimulation protocol for each target. This file is loaded through a bus interface, such as, e.g., a LIGHTNING connector, a micro-USB connector, a USB-C connector, and the like, and is derived through acoustic simulations performed on a brain image set of the user wearing neuromodulation device 110. The simulation maps patient's target brain regions relative to ultrasound-emitting elements 142 of each ultrasound transducer array 140 and appropriately phase corrects each element timing such that a beam focuses on the target.

Referring to FIG. 1A, a stimulation control computing environment disclosed herein also comprises a computing device 250 and an algorithmic framework including one or more processors and a plurality of software and hardware components (including a digital analog converter, function generator, and hard drive) configured to execute program instructions or routines to perform the data processing and performance functions that controls the operability of a neuromodulation device disclosed herein. In certain embodiments, computing device 250 can comprise an offline computing device. In certain embodiments, computing device 250 can comprise a cloud computing environment that is connected to other components of the neuromodulation system 100 over a network, such as the Internet.

An algorithmic framework of stimulation control unit 200 and software elements disclosed herein is part of the one or more systems and methods that apply mathematical functions, models or other analytical and data processing techniques in real-time to ensure a neuromodulation device disclosed herein applies ultrasound stimulation in an appropriate spatial and temporal manner to one or more specific regions of the brain separately and differentially in response to the brain activity data obtained by an EEG electrode.

It should be noted that processing of data and algorithms described herein may be performed by system components implemented in hardware or a combination of hardware and software (see exemplary description of components in FIGS. 1A-1B). As an example, such system and component may include at least one processor, such as a digital signal processor (DSP) or central processing unit (CPU), configured to execute instructions stored in memory for performing the functions described herein. In some embodiments, ASIC or gate arrays, such as field-programmable gate arrays (FPGAs), may be used to implement any of the functions described herein. Various configurations of circuitry for the processing of data and algorithms described here are possible.

Conventional approaches for treating depression have typically focused on cortical regions, such as the dorsolateral PFC, subcallosal cingulate, and anterior cingulate, and/or the mood-regulating ventral capsule/nucleus accumbens. While some results of these conventional approaches showed promise, longitudinal follow-up studies have not supported these treatment modalities, which show that many of the mood-regulating effects of these treatment modalities are likely attributable to placebo effects. Accordingly, there is a need for new treatment modalities and an identification of more effective neural targets. In isolation, the ANT and DMT are not obvious targets for the treatment of depressive symptomology. The ANT is believed to be predominantly involved in spatial and episodic memory via inputs from the hippocampus, and attention and executive processing via bidirectional connections with the prefrontal cortex. Likewise, the DMT is heavily involved in working memory and higher order executive functioning. In fact, evidence from DBS stimulation suggests the ANT may be contraindicated for depression. Subjects from a controlled, randomized trial of 109 patients (SANTE trial) who received DBS of the ANT for the treatment of refractory epilepsy were observed 5 years later. While reductions in seizure frequency persisted over time, depression was observed as a notable adverse event. Depression was reported in 37.3% of patients. Of these, 34% had no prior history of depression. Moreover, an analysis 7 years after the study commencement found a patient-reported subjective increase in depression in the ANT-implanted+DBS stimulated group, in comparison to the ANT-implanted, non-stimulated group.

However, FUS neuromodulation, and more particularly low intensity focused ultrasound (LIFU) have highly distinct mechanisms from DBS, which lead to significant differences in treatment efficacy. Anatomical regions exist as part of broader neural circuits in the brain. For example, thalamic subregions serve as nodes both within the thalamus itself and as part of larger neural networks, including the DMN. Investigating the network functionality of the ANT more closely, a role in emotion regulation may exist through extensive connections with limbic system structures, including the amygdala and hippocampus. Furthermore, the ANT regulates autonomic responses associated with emotional states, such as heart rate and hormonal secretion, via connections with brainstem nuclei involved in autonomic control. Interestingly, dysfunction of the ANT has been linked to various psychiatric disorders marked by emotional dysregulation, including depression and anxiety disorders such post-traumatic stress disorder (PTSD). The multifaceted functions of the ANT in combination with its position in complex neural networks highlight its potential for modulation for the treatment of cognitive and psychiatric disorders.

Major depressive disorder (MDD) is a leading cause of disability worldwide with up to one-third of cases being treatment resistant. Symptom heterogeneity suggests variability across affected brain networks, prompting efforts to personalize circuit-based neuromodulatory interventions. For example, personalized deep brain stimulation (DBS) has been achieved by selecting different treatment targets based on phenotypes or by mapping stimulation responses. However, DBS is invasive, and the stability of optimal long-term treatment may be challenged by dynamic neuroplastic adaptations. On the other hand, noninvasive approaches, such as transcranial magnetic stimulation (TMS), have shown promise in modulating putative mood networks but are unable to target deeper subcortical regions.

In contrast, transcranial low-intensity focused ultrasound (LIFU) is an emerging, non-invasive method with millimeter spatial specificity and a unique ability to achieve deep subcortical neuromodulation. LIFU can reversibly modulate brain networks and confer durable behavioral effects. Preliminary studies suggest that LIFU applied to classical TMS and DBS targets may yield improvements in anxiety, worry, avoidance and mood.

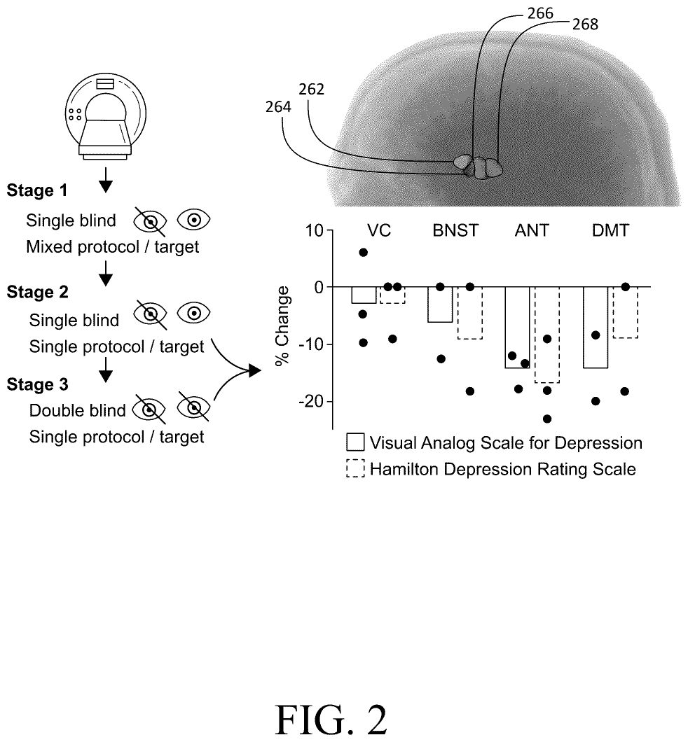

To examine whether dynamically steered LIFU could be used to identify a personalized therapeutic subregions for treatment of MDD, LIFU was delivered using the head-worn neuromodulation device 110, as described with reference to FIGS. 1A-1B. Referring to FIG. 2, a 46 year old subject with treatment resistant depression underwent LIFU interventions to assess target regions that have therapeutic potential based on self-report and objectively investigate the effects of several target brain areas using neuroimaging. The subject's major depression began ten years prior, characterized by anhedonia, apathy, poor energy, poor concentration, guilt, and hopelessness. He had failed a variety of oral agents and psychotherapy. He had prior transient positive effects from both electroconvulsive therapy and TMS, but discontinued treatment due to rapid relapses, cognitive decline, and other side effects, including headache.

As shown in FIG. 2, the subject underwent a three-phase assessment, which included an exploratory phase (Phase 1), candidate-region testing phase (Phase 2), and top-candidate testing with neuroimaging phase (Phase 3). Following the exploratory phase, three potentially therapeutic candidate regions were selected for serial testing: the ventral capsule 262, the bed nucleus of stria terminalis (BNST) 264, the anterior nucleus of the thalamus (ANT) 266, and the mediodorsal thalamus (DMT) 268. Testing of each region was followed by a 24-hr washout period. The top candidate region from serial testing was then compared to an unfocused control stimulation, and resting-state fMRI data were collected post-stimulation.

Behavioral outcomes were collected using visual-analog scales (VAS) of depression and the 6-item Hamilton Depression Rating Scale (HAMD-6). LIFU was delivered using the head-worn neuromodulation device 110. In one embodiment, the candidate regions were sonicated using the same stimulation parameters: a 500 kHz fundamental frequency, a 25 Hz pulse repetition frequency (PRF), a 13% duty cycle (DC), and 300 s (5 min) pulse train duration. It should be understood that in other embodiments the fundamental frequency, the pulse repetition frequency, the duty cycle and the pulse train duration may be varied. For example, a fundamental frequency between approximately 100 kHz and approximately 10 MHz may be selected, a pulse repetition between approximately 10 Hz and approximately 100 Hz may be selected, a duty cycle between approximately 5% and 25% may be selected, and a pulse train between approximately 2 minutes and 10 minutes may be selected.

Returning to FIG. 2, the patient was stimulated bilaterally, alternating between left and right lateralized regions every 15 minutes, i.e. with a 10 min interval between each 5 min pulse-train. The unfocused control stimulation constitutes the same acoustic energy at the array surface but does not include a deep brain focal pattern, allowing for the same acoustic and peripheral experience as a focal stimulation paradigm. Eight total pulse trains (four left and four right lateralized) were performed to complete a full session with each performed within a single day.

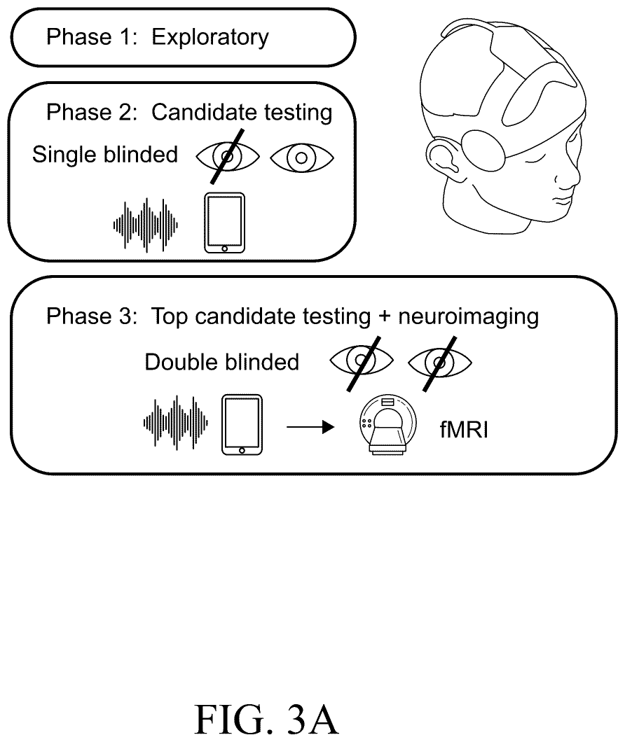

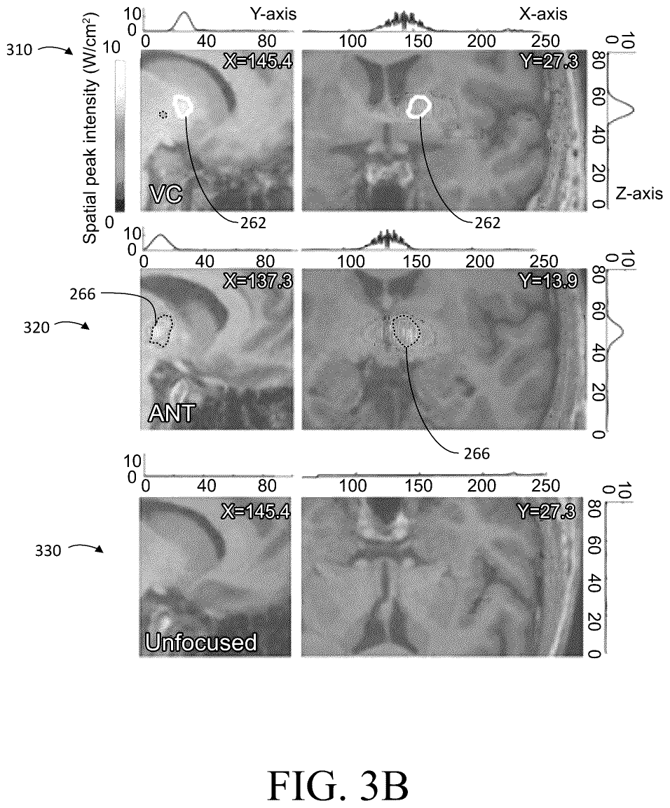

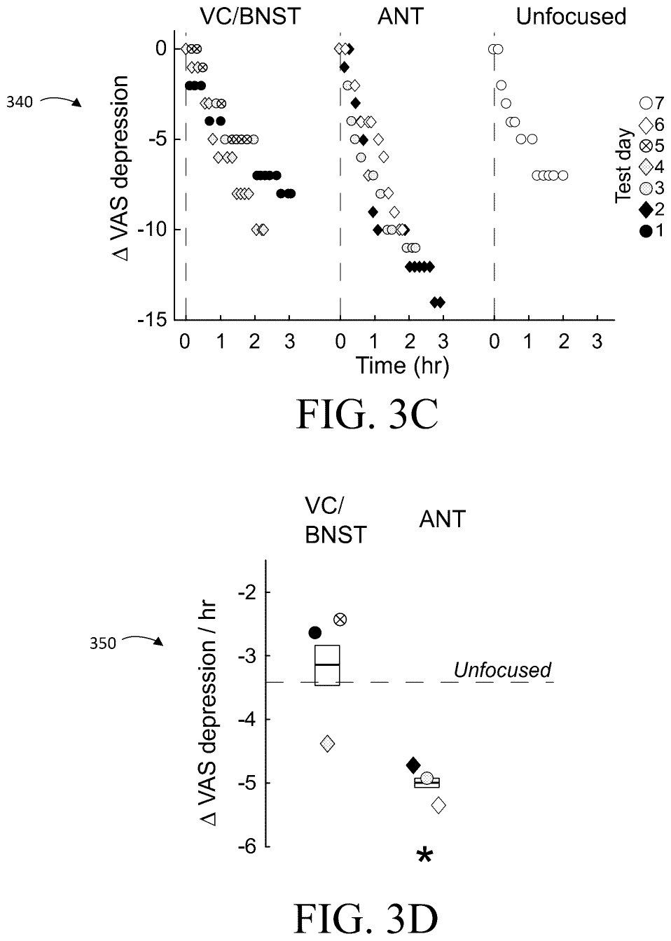

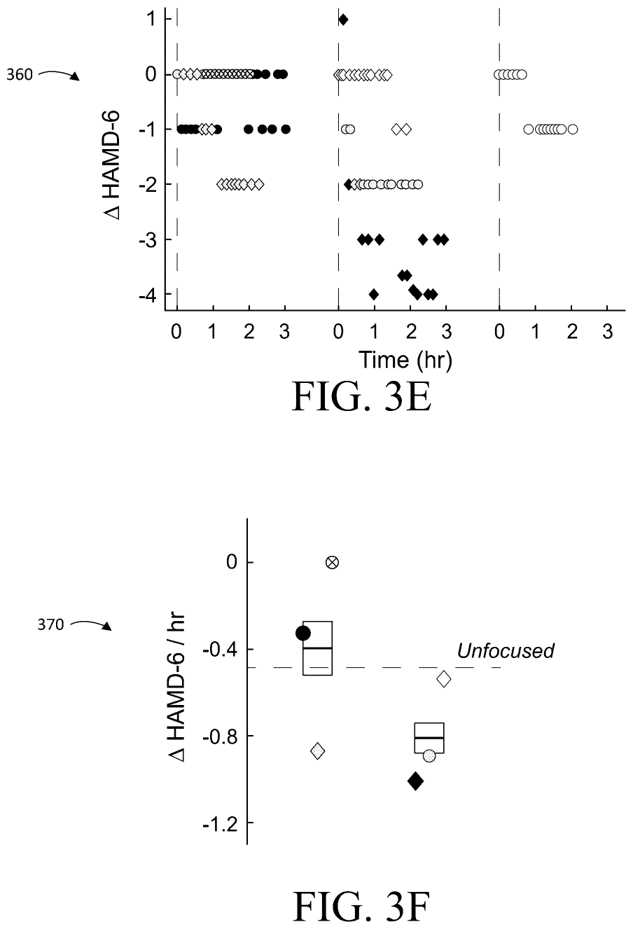

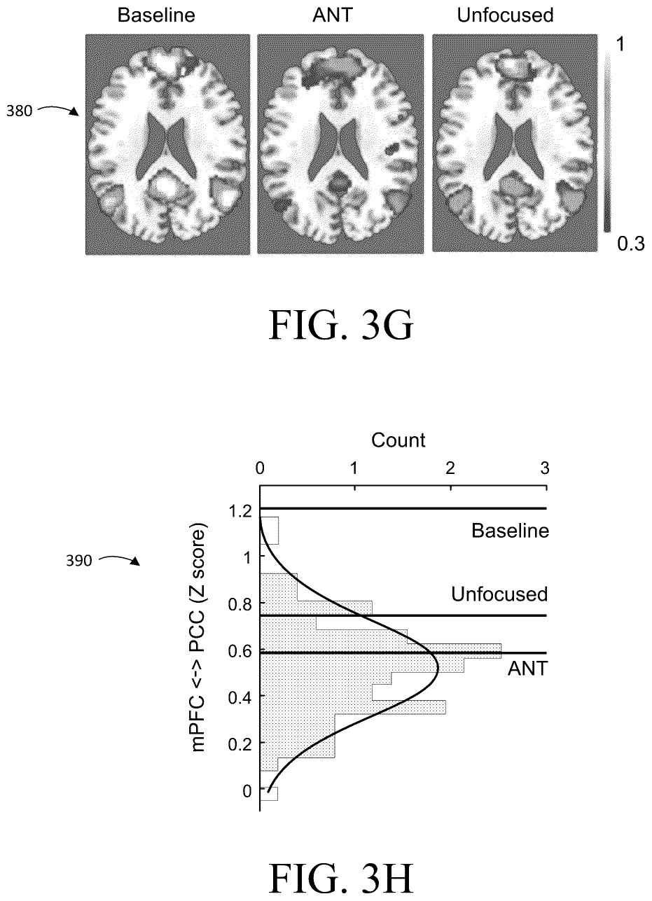

FIG. 3B shows the overlay of the right-lateralized simulated ultrasound intensities overlaid on the sagittal and coronal T1-weighted MR images for the VC 262 (image 310), ANT 266 (image 320), and unfocused conditions (image 330). Simulated ISSPA ranged from 8.3 to 10.6 W/cm2 at the target for active conditions and <2 W/cm2 at any given target for the unfocused control. Note that the VC 262 and ANT 266 masks are non-overlapping; the mask of each of the other active condition is not seen in the respective cross-section, except that the edge of the ANT target 266 is seen in the sagittal view of the VC 262 image.

The intermittent LIFU protocol was well tolerated by the participant without any adverse effects. A post-stimulation MRI safety scan did not reveal any structural change, including edema or hemorrhage. Over the course of serial stimulations, a reduction in VAS-depression and HAMD-6 was observed across all stimulation conditions. For example, FIG. 3C illustrates in graph 340 the change in VAS-depression scale as a result of focusing on the VC 262 and BNST 264 on the left column, ANT 266 in the middle column, and unfocused ultrasound on the right column as a control. FIG. 3D illustrates in graph 350 the averaged change in VAS depression and FIG. 3F illustrates in graph 370 the HAMD-6 score per hour for each condition: VC/BNST (left) and ANT (right). FIG. 3E illustrates in graph 360 effects on subject depression scores after exposing various target brain regions to focused ultrasound. The unfocused stimulation condition is given by the dotted line in graph 370 of FIG. 3F. The ANT stimulation condition statistically reduced the averaged VAS depression per time, as compared to the unfocused control (p=0.0127; one sample t-test). When compared to the unfocused control condition, ANT stimulation further reduced VAS-D scores over time (t(2)=−8.87, p=0.013, one sample t-test), in contrast to VC/BNST stimulation (t(2)=−2.6, p=0.152) as illustrated in FIG. 3D. Similar trends were observed in the HAMD-6 scores but without statistical significance (see FIGS. 3E-3F). An example spontaneous verbal report following an exploratory ANT stimulation day included: “I think I'm having less obsessive-compulsive thoughts . . . when I start getting on myself for something it's just hard to get off, but I feel like I've been moving through my thoughts a little bit better,” suggesting a reduction in ruminative thinking.

Subsequently, the effects of stimulation on functional connectivity within the default mode network (DMN) was evaluated. As discussed above, the default mode network is a network of brain regions implicated in self-reflection and rumination. At baseline, resting-state DMN connectivity was hyperconnected in this subject as compared to connectivity seen in a distribution of aged-matched healthy individuals, as shown in FIG. 3H (n=84; age, mean=43.96 y, SD=18.3 y; sex, 41 F). Resting-state fMRI following double-blinded ANT 266 stimulation also showed a reduction in DMN connectivity when compared to the atypically high levels of connectivity seen in the baseline condition. Smaller reductions in DMN connectivity compared to baseline were also seen following the unfocused condition as shown in FIGS. 3G-3H. More particularly, FIG. 3G shows in visualization 380 of default mode network (DMN) connectivity based on a medial prefrontal cortex seed and shows that ANT stimulation reduces DMN connectivity as compared to the baseline and unfocused conditions (Fisher-transformed correlation coefficient units). FIG. 3H shows in graph 390 the normalized probability density function of anterior-posterior DMN connectivity in a group of healthy controls (histogram bars) and overlaid black normal distribution curve. The bars indicate baseline/pre-stimulation, ANT stimulation, and unfocused stimulation, respectively. Note that unfocused condition occurred the following day, i.e. 24-hrs, after ANT stimulation. Notably, subjective sleepiness was not affected by stimulation, rated as a 4 on the Stanford Sleepiness Scale across the ANT and unfocused conditions, and is therefore unlikely to explain the changes in DMN connectivity.

As shown in FIGS. 3A-3H, LIFU directed to the ANT 266 has the potential to elicit a subjective reduction in depression symptoms that is associated with decreases in DMN connectivity assessed through resting-state fMRI. In contrast, the VC 262 and BNST 264 targets did not yield a statistically significant reduction in symptom scores. Furthermore, it is shown that an intermittent stimulation paradigm can progressively shift mood symptoms over time, when compared to the trajectory of an unfocused control condition. Specifically, symptom trajectories suggests improvement over the course of hours, as compared to other non-invasive methods such as TMS, which evolve over days to weeks. In contrast to VC/BNST, ANT 266 is not a common target for DBS in MDD but has been implicated in emotional regulation by way of its direct connectivity with anterior cingulate and prefrontal cortex, (e.g., the medial and orbital frontal cortex). Counterintuitively, DBS stimulation targeting the bilateral ANT 266 has been reported to increase the rates of depressive adverse events. In contrast to the application of DBS, the results show that LIFU of ANT 266 is associated with improvement in depressive symptoms, highlighting potentially distinct underlying mechanisms of these two neuromodulation modalities. Notably, this participant went on to separate clinical trial involving intracranial DBS mapping, for which VC/BNST yielded the strongest acute mood responses.

In this experiment, symptom improvement associated with ANT-LIFU in this individual was causally related to associated decreases in DMN connectivity, which was shown to be hyperconnected in this individual and is generally overactive in MDD. Current depression treatments, ranging from medications (SSRIs, psychedelics) to mindfulness, have been associated with a reduction in DMN connectivity.

In another experiment, it was investigated whether ANT 266 stimulation could significantly modify the default mode network and mood in healthy subjects, which has implications for improving mindfulness, attention, and task execution. The results support the finding that ANT-LIFU stimulation could be used as a preventative tool to stave off depression in vulnerable individuals and populations. The experiment included performing bilateral ANT 266 stimulation for 20 minutes on 9 healthy subjects, using a 25 Hz, 8% duty cycle protocol. We subsequently measured the DMN through fMRI measurements at 40-60 minutes post stimulation onset. 20 minutes of resting state fMRI were collected. It should be understood that in other embodiments the fundamental frequency, the pulse repetition frequency, the duty cycle and the pulse train duration may be varied. For example, a fundamental frequency between approximately 100 kHz and approximately 10 MHz may be selected, a pulse repetition between approximately 10 Hz and approximately 100 Hz may be selected, a duty cycle between approximately 5% and 25% may be selected, and a pulse train between approximately 2 minutes and 10 minutes may be selected.

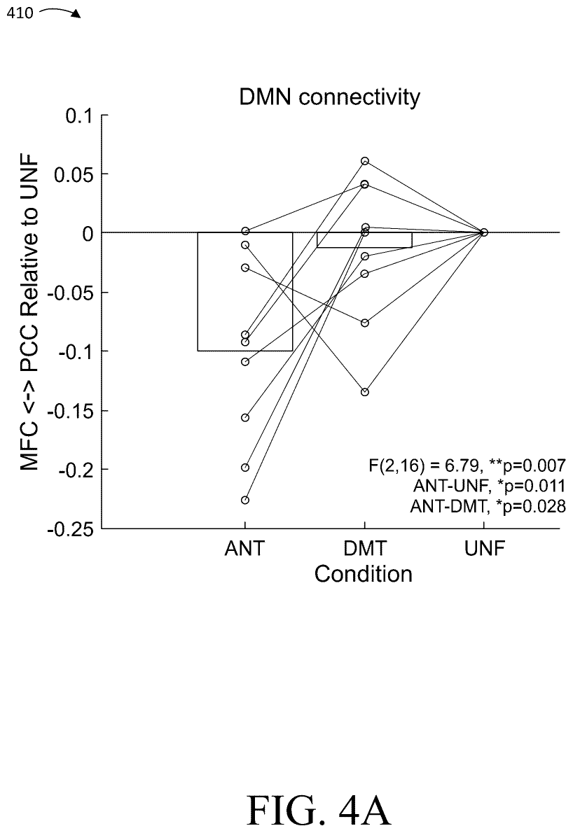

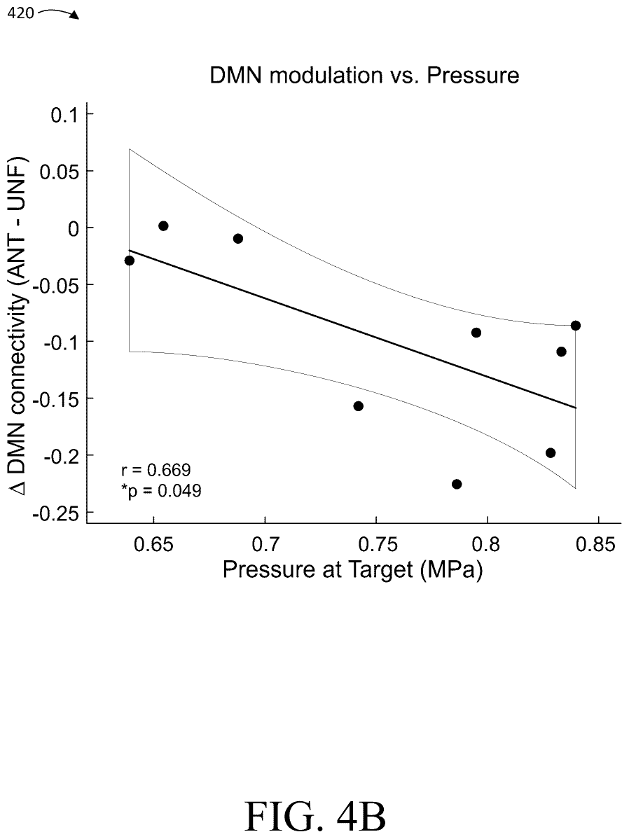

As further illustrated in FIGS. 4A-4B in graph 410 and graph 420, the functional connectivity between the Medial Frontal Cortex (MFC) and Posterior Cingulate Cortex (PCC) was assessed relative to unfocused control within individuals. The relative connectivity was significantly reduced following ANT stimulation as compared to DMT stimulation. The reduction in connectivity was found to be directly related to pressure of the focused ultrasound beam delivered to the target brain region. This suggests that increasing pressure further may enhance reduction in DMN connectivity and improvements in mood and mindfulness. It also suggests that lowering pressure may actually increase connectivity of the DMN, where increased connectivity may be used to reduce bipolar symptoms of mania, or psychopathic behavior where DMN connectivity is too low. It may also be used to increase chance of insightful moments through referential thought processes.

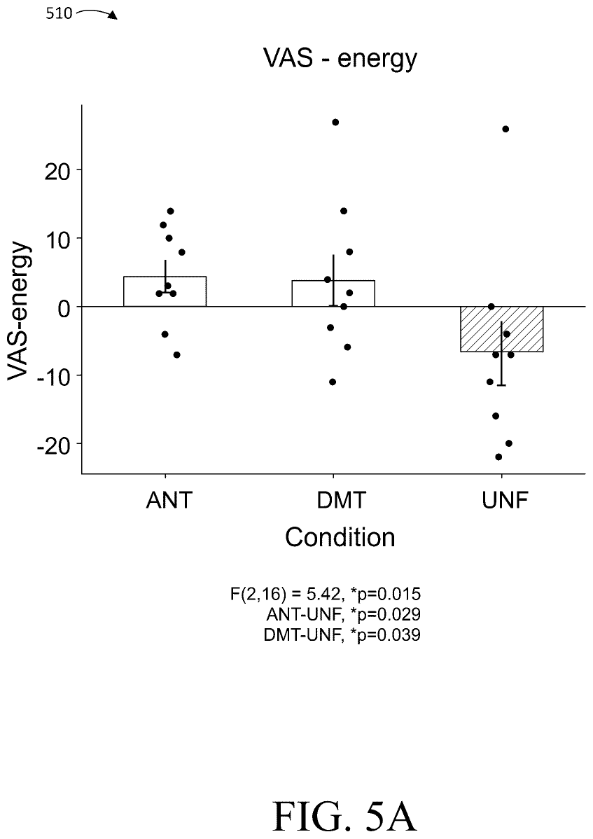

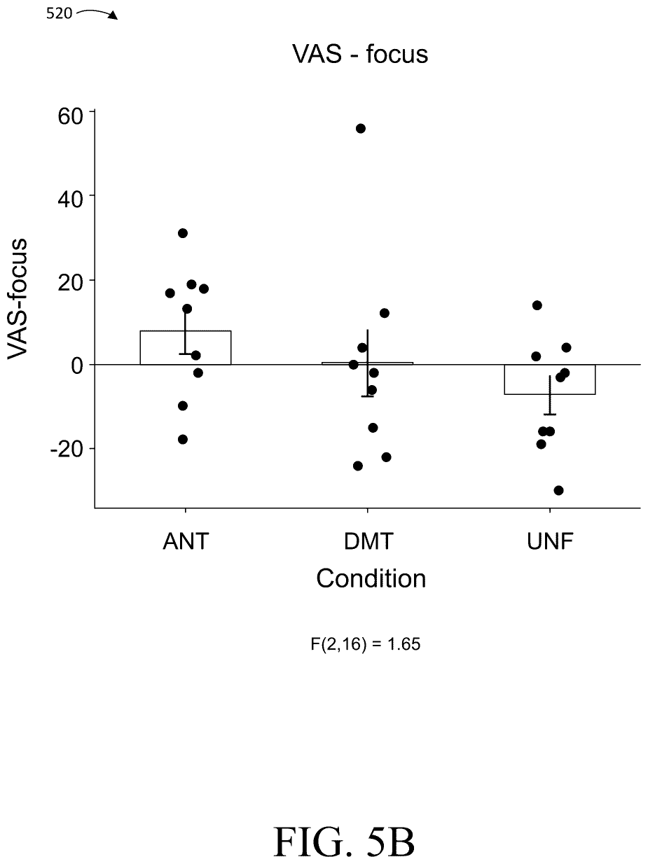

FIGS. 5A-5B illustrate the effects on self-reported ratings of energy and focus in healthy individuals, after an application of LIFU to ANT 266 and DMT 268, with an unfocused control for reference. The energy and focus scales are represented on a visual analog scale and are presented as the change in rating from pre to post stimulation (post-pre). As shown in FIG. 5A in graph 510, stimulation of the ANT 266 provided similar increases to energy in subjects as stimulation of DMT 268. Notably, an increase in focus was found after stimulation of the ANT 266, but not the DMT 268, as illustrated in FIG. 5B in graph 520.

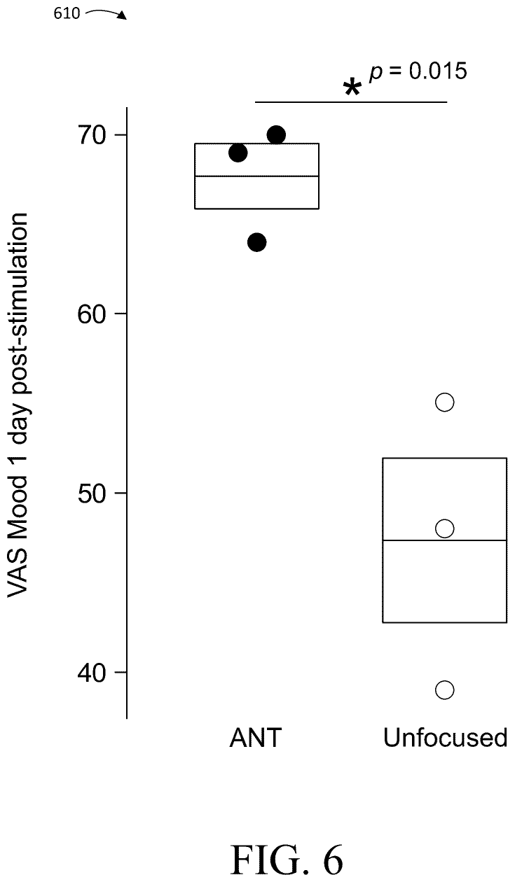

FIG. 6 illustrates in graph 610 the effect of LIFU stimulation applied to ANT 266 with respect to mood, also represented on the visual analog scale. As shown in FIG. 6, LIFU stimulation of ANT 266 is heavily correlated with a marked increase in mood one day post-stimulation.

It should be noted that processing of data and algorithms described herein may be performed by system components implemented in hardware or a combination of hardware and software (see exemplary description of components in FIGS. 1A-1C). As an example, such system a component may include at least one processor, such as a digital signal processor (DSP) or central processing unit (CPU), configured to executes instructions stored in memory for performing the functions described herein. In some embodiments, application-specific integrated circuits (ASICs) or gate arrays, such as field-programmable gate arrays (FPGAs), may be used to implement any of the functions described herein. Various configurations of circuitry for the processing of data and algorithms described here are possible.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

As a further example, variations of apparatus or process parameters (e.g., dimensions, configurations, components, process step order, etc.) may be made to further optimize the provided structures, devices, and methods, as shown and described herein. In any event, the structures and devices, as well as the associated methods, described herein have many applications. Therefore, the disclosed subject matter should not be limited to any single embodiment described herein, but rather should be construed in breadth and scope in accordance with the appended claims.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this description are approximate unless stated otherwise, meaning that the term “about” or “approximately” can be inferred when not expressly stated.

With reference to the use of the word(s) “comprise,” “comprises,” and “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “including” should be interpreted to mean “including but not limited to . . . ” unless the context clearly indicate otherwise.

The term “consisting essentially of” means that, in addition to the recited elements, what is claimed may also contain other elements (steps, structures, ingredients, components, etc.) that do not adversely affect the operability of what is claimed for its intended purpose. Such addition of other elements that do not adversely affect the operability of what is claimed for its intended purpose would not constitute a material change in the basic and novel characteristics of what is claimed.

The term “adapted to” means designed or configured to accomplish the specified objective, not simply able to be made to accomplish the specified objective.

The term “capable of” means able to be made to accomplish the specified objective.

The terminology used herein is for the purpose of describing particular embodiments only and is not intended to be limiting. As used herein, the singular forms “a”, “an” and “the” are intended to include the plural forms as well (i.e. “at least one”), unless the context clearly indicates otherwise.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

Terms such as “at least one of A and B” should be understood to mean “only A, only B, or both A and B.” The same construction should be applied to longer list (e.g., “at least one of A, B, and C”).

What is claimed is:

1. A head-mounted device comprising:
an ultrasound transducer element housing;
one or more ultrasound transducer elements positioned within the ultrasound transducer element housing and configured to be positioned approximate a temporal window of a user when the head-mounted device is worn on a head of the user; and
one or more processors;
a non-transitory memory operatively coupled to the one or more processors and storing instructions that when executed by the one or more processors cause the one or more processors to control the one or more ultrasound transducer elements to:
create at least one ultrasound focus at a target region within the head of the user wearing the head-mounted device; and
deliver focused ultrasound to the target region comprising a frequency between approximately 100 kHz and 1 MHz, thereby modulating a level of connectivity within a default mode network of the user.

2. The head-mounted device of claim 1, wherein the target brain region comprises at least a portion of the thalamus.

3. The head-mounted device of claim 1, wherein the target brain region comprises the anterior thalamus.

4. The head-mounted device of claim 1, wherein modulating the level of connectivity within the default mode network increases present-tense experiential engagement by decreasing the frequency and/or duration of non-present focused thinking.

5. The head mounted device of claim 1, wherein modulating the level of connectivity within the default mode network enhances attention during tasks.

6. The head mounted device of claim 1, wherein modulating the level of connectivity within the default mode network results in a reduction of ruminative thinking.

7. The head mounted device of claim 1, wherein the one or more ultrasound transducer elements comprise:
a first ultrasound transducer element array positioned in a first position within the ultrasound transducer element housing; and
a second ultrasound transducer element array positioned in a second position within the ultrasound transducer element array housing, the second position disposed at an arc length away from the first position;
wherein the non-transitory memory includes further instructions, that when executed by the one or more processors cause the one or more processors to control the one or more ultrasound transducer element arrays to:
cause the first ultrasound transducer element to deliver a first focused ultrasound beam to the target region; and
cause the second ultrasound transducer element to deliver a second focused ultrasound beam to the target region, wherein the first focused ultrasound beam and a the second focused ultrasound beam cross at a predetermined crossing angle at the target region.

8. The head mounted device of claim 1, wherein the non-transitory memory includes further instructions, that when executed by the one or more processors cause the one or more processors to control the one or more ultrasound transducer elements to be pulsed at a pulse rate between approximately 10 Hz and approximately 30 Hz.

9. The head mounted device of claim 1, wherein the non-transitory memory includes further instructions, that when executed by the one or more processors cause the one or more processors to control the one or more ultrasound transducer elements to deliver focused ultrasound with a duty cycle of less than 15%.

10. The head mounted device of claim 1, wherein the non-transitory memory includes further instructions, that when executed by the one or more processors cause the one or more processors to control the one or more ultrasound transducer elements to deliver a first pulsing pattern for a period of at least five minutes.

11. The head mounted device of claim 1, wherein modulating the level of connectivity within the default mode network cause a reduction in symptoms of major depressive disorder, obsessive-compulsive disorder, anxiety, post-traumatic stress disorder, attention deficit hyperactivity disorder, autism spectrum disorder, sleep disorder, or any combination thereof.

12. A method, comprising:
causing, by one or more processors in operative communication with one or more ultrasound transducer elements positioned within an ultrasound transducer element housing of a head-mounted device, the one or more ultrasound transducer elements to create at least one ultrasound focus at a target region within a head of a user wearing the head-mounted device; and
delivering focused ultrasound to the target region comprising a frequency between approximately 100 kHz and 1 MHz, thereby modulating a level of connectivity within a default mode network of the user.

13. The method of claim 12, wherein the one or more ultrasound transducer elements are positioned approximate a temporal window of the user when the head-mounted device is worn on the head of the user.

14. The method of claim 12, wherein the target brain region comprises the anterior thalamus.

15. The method of claim 12, wherein modulating the level of connectivity within the default mode network increases present-tense experiential engagement by decreasing the frequency and/or duration of non-present focused thinking.

16. The method of claim 12, wherein modulating the level of connectivity within the default mode network enhances attention during tasks.

17. The method of claim 12, wherein modulating the level of connectivity within the default mode network results in a reduction of ruminative thinking.

18. The method of claim 12, wherein the one or more ultrasound transducer elements comprise:
a first ultrasound transducer element positioned in a first position within the ultrasound transducer element housing; and
a second ultrasound transducer element positioned in a second position within the ultrasound transducer element housing, the second position disposed at an arc length away from the first position;
wherein the method further comprises:
causing, by the one or more processors, the first ultrasound transducer element to deliver a first focused ultrasound beam to the target region; and
causing, by the one or more processors, the second ultrasound transducer element to deliver a second focused ultrasound beam to the target region, wherein the first focused ultrasound beam and the second focused ultrasound beam cross at the anterior thalamus.

19. The method of claim 12, further comprising causing, by the one or more processors, the one or more ultrasound transducer elements to be pulsed at a pulse rate between approximately 10 Hz and approximately 30 Hz.

20. The method of claim 12, further comprising controlling, by the one or more processors, the one or more ultrasound transducer elements to deliver focused ultrasound with a duty cycle of less than 15%.

21. The method of claim 12, further comprising controlling, by the one or more processors, the one or more ultrasound transducer elements to deliver a first pulsing pattern for a period of at least five minutes.
